SEQUENCE LISTING (2 sequences)

SEQ ID NO: 1 - DNA, 777 bp, Pan troglodytes
atgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttaactatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcaccaaaataaatcatctttattaagtagatgaaacattaactctaactgtgacaaagaagaccacagatagttatcttttaattacagaagagtttcttaacttatttttgtaagtttttattgtgtaagtttataatgcaggggaagtactactcctcaaatgttgagggaagcttccataacatcgatgactgacttcatggcagtaattctcagctgtagttgcataagcattgctcaagaggaaaatccaaaagtgcagcagg

SEQ ID NO: 2 - protein, 159 aa, Pan troglodytes
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALTIWCPGYSETQINATQAMKKRRKRKVTTNKCLEQVSQLQGLWRRFNRPLLKQQ

(19) United States
(12) Patent Application Publication
(10) Pub. No.: US 2010/0021486 A1
(43) Pub. Date: Jan. 28, 2010

(54) TSLP VACCINE FOR THE TREATMENT OF TH2 MEDIATED INFLAMMATORY CONDITIONS

(75) Inventor: Lars Hellman, Vardkasvagen (SE)

Correspondence Address: BACON & THOMAS, PLLC, 625 SLATERS LANE, FOURTH FLOOR, ALEXANDRIA, VA, 22314-1176, US

(73) Assignee: Theravac Pharmaceuticals AB, Vardkasvagen 1 (SE)

(21) Appl. No.: 12/312,812

(22) PCT Filed: Nov. 26, 2007
(86) PCT No.: PCT/SE2007/001037

§ 371 Date: Oct. 1, 2009

(30) Foreign Application Priority Data
Nov. 28, 2006 (SE) 0602550-6

(51) Int. Cl. A61K 39/00; C12P 21/02

(52) U.S. Cl. 424/185.1; 435/69.3

(57) ABSTRACT

The invention relates to compositions for the treatment of TH2 mediated inflammatory conditions. The compositions are vaccines comprising a non-primate TSLP or fragments thereof to be used as a vaccine to treat humans with excessive mediated inflammatory conditions. For the treatment of TH2 mediate inflammatory conditions in other non-human mammals the TSLP of the species to be vaccinated is being used as antigen.

The present invention relates to methods designed to alleviate the symptoms or prevent the induction of TH2 mediated inflammatory conditions by targeting the cytokine TSLP (Thymic stromal lymphopoetin). Although the invention generally relates to a vaccine for use in a mammal, preferred embodiments thereof relates to vaccines for the use in human, dog, cat or horse, the invention will be described below generally, and with reference to such vaccines for human, feline, equine or canine use.

### BACKGROUND OF THE INVENTION

During the past few decades several diseases caused by the malfunction of the immune system have become major challenges of modem day medicine. One such area is allergic diseases. Allergies have in man become almost epidemic during the past 20-30 years and atopic, or IgE-mediated allergies, are the dominating form. Allergies are also a major problem for many domestic animals like dogs, cats and horses. However, the involvement of IgE is here less well documented. Common types of atopic allergies include in man, fur allergies, hay fever, dust mite allergies, insect venom allergies, extrinsic asthma and many types of food allergies. In addition, many of our domestic animals suffer from allergies directed against similar allergens. Allergies has been estimated to affect almost 30% of the human population.

Allergic diseases are caused by malfunctions in our immune system. Several of the cytokines regulating normal immune responses against various pathogens appear also to be directly involved in the allergic disease processes. Cytokines, or growth and differentiation factors of importance for the regulation of our immune system may thereby serve as potential targets for intervention.

One particular difficult problem in veterinary medicine is severe atopic dermatitis in dogs. Almost 50% of all visits to the veterinarians are due to skin problems where atopic dermatitis is the dominating factor. In human medicine a particularly difficult condition is instead severe asthma. For the most severe cases non of the existing treatment regiments show sufficient clinical effect. In the situation with severe atopic dermatitis in dogs many of these dogs have to be removed. We have here a great unmet medical need.

We have for many years been working on a potential treatment strategy against IgE mediated allergies in man and domestic animals, vaccination against IgE [1-5]. However in cases where IgE levels are exceptionally high as in dogs [6], this strategy has its clear limitations. These limitations are primarily due to strong tolerizing effects induced by the high concentrations of circulating IgE. In addition, it is difficult to obtain good clinical effects when large amounts of the target molecule have to be removed. We therefore saw it as almost impossible to reach our goal in dogs by vaccinating against IgE [6]. New innovative strategies had to be developed. We here present one potential solution to the problem, vaccination against one important cytokines regulating humoral immunity the thymic stromal lymphopoietin (TSLP).

TSLP (thymic stromal lymphopoietin) has been described as the master switch of allergic inflammation [7]. TSLP is an IL-7 like cytokine that in humans is produced predominantly by epithelial cells and mast cells. This cytokine stimulates mDC to promote the differentiation of naïve CD4+ T cells into TH2 type effector cells [7]. These TH2 cells produce the allergy promoting cytokines IL-4, IL-5, IL-13 and TNF-α, but not IL-10 and IFN-γ [8]. Recent findings also indicate that TSLP activated DCs play important roles not only in TH2 priming, but also in the maintenance and further polarization of TH2 central memory cells in allergic diseases [9]. Keratinocytes in atopic dermatitis lesions has been shown to express high levels of TSLP and may thereby be a key cytokine in the development of atopic dermatitis [8]. Mice engineered to over-express TSLP in skin also develop atopic dermatitis and a dramatic increase in circulating TH2 cells and elevated serum IgE [10]. TSLP is however not only expressed in peripheral tissues but also by Hassall's corpuscles within the human thymus. Here, TSLP is involved in instructing thymic DCs to convert high affinity self-rective T cells into CD4+CD25+ FoxP3+ regulatory T cells. Concerning its receptor, the hetrodimeric TSLP receptor appears to be exclusively expressed by myeloid dendritic cells (mDCs).

TSLP appears to be one of the key regulators of TH2 mediated inflammatory conditions and may thereby serve as a very interesting target for therapeutic intervention [11]. I here describe a vaccination strategy, which is aimed to modulate excessive TH2 mediated inflammatory conditions by targeting TSLP. This vaccine may become a new important step in the management of severe asthma in humans and atopic dermatitis in dogs.

### THE PRIOR ART

Patent applications describing the use of monoclonal antibodies or soluble receptors for blocking the activity of human TSLP has been filed. These strategies for targeting TSLP is dependent on injections of highly purified recombinant protein every two to four weeks possibly for the rest of the life of the patient. A vaccine, as described in this application, could here serve as a major improvement over prior art due to that it rely on injections of recombinant protein in a much smaller scale, maybe as little as 10 000 times lower amount compared to the amount needed for treatment with monoclonals or soluble receptor. A vaccines most likely also needs to be administrated one to four times a year as compared to the much more frequent administrations of monoclonals or soluble receptor as described above. The use of a non-human primate sequence may also increase the immunogeneicity of the resulting vaccine in humans. For therapeutic applications other than vaccines the non-modified version of TSLP (the human protein) has to be used in order not to elicit an immune response against the protein, which may substantially reduce the effect of the injected recombinant protein.

### OBJECT OF THE INVENTION

The object of the invention is to provide a convenient and cost effective method to treat various inflammatory conditions caused by excessive TH2 type immunity. Treatment with a vaccine with a fusion protein consisting of TSLP (or parts of TSLP) and a foreign carrier protein reduces the levels of free TSLP and thereby reduces the symptoms caused by excessive release of this cytokine.

### SUMMARY OF THE INVENTION

The above object is achieved according to the invention by a vaccine, which is characterized by containing a protein having the entire amino acid sequence of TSLP from a non-human primate (if to be used in humans) or a segment larger than 5 amino acids of said amino acid sequence, in its original or multimerized form. When treating domestic animals the TSLP sequence from the species to vaccinated is instead being used. The protein may optionally be coupled to one or more heterologous carrier proteins and by optionally containing an adjuvant. The TSLP molecule to be used in the vaccine antigen can alternatively be modified by one or a few point mutations to block its binding to its receptor. The protein will thereby not have TSLP activity when injected in the host but will still maintain its potency to induce antibodies that also react with native TSLP.

### BRIEF DESCRIPTION OF THE FIGURES

FIG. 1 shows the nucleotide and the corresponding amino acid sequence of chimpanzee TSLP.

### DESCRIPTION OF THE INVENTION

Anti TSLP antibodies are produced in the host by active immunization, so called vaccination. By injecting a modified TSLP molecule into the host the immune system of the host produces a polyclonal antibody response directed against its own TSLP thereby down regulating the effects of its potentially excessive TSLP production. It is of major importance to modify the antigen so that the immune system of the host recognize the modified self-protein as a non-self protein. This can be achieved by covalent coupling of non-self amino acid regions to TSLP or a selected region of TSLP from the species to be treated. The peptides within the non-self region then attract and activate non-tolerized T cells, which give help for the potentially auto-reactive B cells.

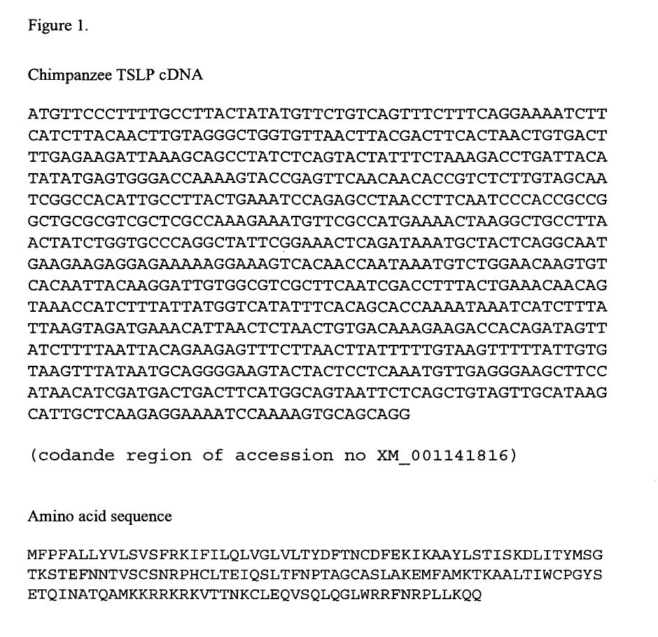

There are at least four possible strategies to do this modification of the self-protein. One method is to produce a fusion protein between a non-self protein, and the entire or a selected fragment of more than 5 amino acids of self-TSLP in a prokaryotic or eukaryotic expression system. The open reading frame of TSLP, as exemplified by canine and human TSLP in FIG. 1, is then first being cloned into a bacterial, fungal or eukaryotic fusion protein vector. This fusion protein construct is then transfected into a mammalian or prokaryotic host for production of the desired fusion protein. The fusion partner can here be any non-self protein of any size from 10 amino acids to several hundred kD. However, it is usually favorable to use a fusion partner of approximately the same size as the self-protein.

Alternatively, an immunodominant peptide can be inserted into the TSLP structure giving rise to a fusion protein with self-TSLP sequences on both sides of the foreign peptide.

As a third alternative, a non-modified TSLP can be produced in a mammalian or prokaryotic host or host cell line and then covalently attached to a carrier protein by chemical coupling.

The fourth alternative, which in our mind less favorable, is to produce selected regions of the TSLP sequence as synthetic peptides and then to couple these peptides to a foreign carrier molecule by chemical coupling. This fourth alternative usually results, after injection into the patient, in antibody responses that show low binding activity against the native properly folded protein and thereby in lower clinical effect.

Following production the vaccine antigen is then purified and tested for pyrogen content and potential content of other contaminants. In order to obtain sufficiently strong immune response against the self-epitopes the vaccine antigen is then (optionally) mixed with an adjuvant before injection into the patient. After administration in the patient the vaccine induces an immune response against the vaccine antigen. Due to the presence of self-epitopes in the vaccine antigen this protein also induces an antibody response against the target molecule, here TSLP, thereby reducing the levels of this protein in the patient.

### REFERENCES

[] Hellman L. Profound reduction in allergen sensitivity following treatment with a novel allergy vaccine. Eur J Immunol 1994;24(2):415-20.

[2] Hellman L. Is vaccination against IgE possible? Adv Exp Med Biol 1996;409:337-42.

[3] Hellman L, Carlsson M. Allergy vaccines: A review of developments. Clin Immunotherapeutics 1996;6(2): 130-42.

[4] Hellman L. Vaccines against allergies. In: Perlmann P, Wigzell H, editors. Handbook of Experimental Pharmacology, Vol133, Vaccines. Berlin, Springer-Verlag, 1999: 499-526.

[5] Vernersson M, Ledin A, Johansson J, Hellman L. Generation of therapeutic antibody responses against IgE through vaccination. Faseb J 2002;16(8):875-7.

[6] Ledin A, Bergvall K, Salmon-Hillbertz N, Hansson H, Andersson G, Hedhammar Å, et al. Generation of therapeutic antibody responses against IgE in dogs, an animal species with exceptionally high plasma IgE levels. Vaccine 2006;24, 66-74.

[7] Liu Y J. Thymic stromal lymphopoietin: master switch for allergic inflammation. J Exp Med 2006;203(2):269-73.

[8] Soumelis V, Reche P A, Kanzler H, Yuan W, Edward G, Homey B, et al. Human epithelial cells trigger dendritic cell mediated allergic inflammation by producing TSLP. Nat Immunol 2002;3(7):673-80.

[9] Wang Y H, Ito T, Wang Y H, Homey B, Watanabe N, Martin R, et al. Maintenance and polarization of human TH2 central memory T cells by thymic stromal lymphopoietin-activated dendritic cells. Immunity 2006;24(6):827-38.

[10] Yoo J, Omori M, Gyarmati D, Zhou B, Aye T, Brewer A, et al. Spontaneous atopic dermatitis in mice expressing an inducible thymic stromal lymphopoietin transgene specifically in the skin. J Exp Med 2005;202(4):541-9.

[11] Huston D P, Liu Y J. Thymic stromal lymphopoietin:a potential therapeutic target for allergy and asthma. Curr Allergy Asthma Rep 2006;6(5):372-6.

1-6. (canceled)

7. A vaccine comprising a Thymic stromal lymphopoetin (TSLP) (Seq. ID No. 1) or at least one fragment thereof from a non-human primate or another non-human mammal and pharmaceutically acceptable adjuvants.

8. A vaccine according to claim 7, wherein at least one fragment of TSLP in its original or multimerized form is coupled to a carrier molecule.

9. A vaccine according to claim 7, wherein the TSLP is non-human primate, canine, feline or equine TSLP.

10. A vaccine according to claim 8, wherein the TSLP is non-human primate, canine, feline or equine TSLP.

11. A TSLP vaccine according to claim 7 containing an effective amount of said TSLP or fragment thereof to alleviate symptoms or prevent induction of TH2 mediated inflammatory conditions.

12. A process for the preparation of a vaccine according to claim 7 characterized by cloning of cDNA, or a genomic sequence of TSLP or a region encoding more than 5 amino acids thereof; ligating the same into a suitable vector, transforming the vector into an eukaryotic or prokaryotic host cell for the production of a fusion protein, containing the entire TSLP sequence or a region thereof its original or in an mutated or multimerized form, and purifying and optionally mixing the obtained fusion protein with a suitable adjuvant.

13. The use of a fusion protein consisting of the entire or parts of TSLP from the species to be treated (or a closely related species) and a foreign carrier protein for production of a vaccine for medical use.
